(19) United States
(12) Patent Application Publication
(10) Pub. No.: US 2011/0092821 A1
(43) Pub. Date: Apr. 21, 2011

(54) DEVICE FOR MEASURING THE MECHANICAL PROPERTIES OF THE SKIN WITHOUT CONTACT WITH THE MEASUREMENT ZONE

(75) Inventors: Arnaud Fournial, Paris (FR); Philippe Mondon, Paris (FR); Sébastien Fache, Paris (FR); Claire Mas Chamberlin, Chevreuse (FR)

(73) Assignee: SEDERMA S.A.S., LE PERRAY EN YVELINE (FR)

(21) Appl. No.: 12/736,991

(22) PCT Filed: May 29, 2009
(86) PCT No.: PCT/IB2009/052269

§ 371 Date: Nov. 30, 2010

(30) Foreign Application Priority Data
May 30, 2008 (FR) 0853559

(51) Int. Cl. A61B 6/00; A61B 5/103

(52) U.S. Cl. 600/473; 600/587

(57) ABSTRACT

The invention relates to a device for measuring the mechanical properties of the skin which is free from all parameters interfering with measurement. The device comprises: a) a pneumatic assembly (16) for delivering compressed air to apply a stress to the surface of the skin undergoing measurement, b) an electronic assembly (6) for contact-free measurements on said surface of the skin subjected to the stress, and c) a control software (19) for controlling the electronic assembly (6) and for processing measurement data in order to deduce the mechanical properties of the skin.

This invention relates to a device for measuring the mechanical properties of the skin. More particularly, the device according to the invention enables a high-frequency measurement of the mechanical properties of the skin that is free from all parameters interfering with measurement. Methods of measurement of the mechanical properties of the skin have been known for many years and their use, to evaluate, in particular, the state of healthy skin and the changes induced by cosmetic products have been widely reported in the scientific literature (Agache and Humbert, 2004).

The methods employ stretching, torsion, suction, compression or percussion implemented by various systems such as: the Cutometer (EP0362616 A1) and Ballistometer (FR 2 533 030 A1), etc.

However, in addition to the stress exerted on the skin, all the systems have the disadvantage of making direct contact with the measurement zone, thus giving rise to interfering parameters. The device in direct contact with the measurement zone exerts pressure on the skin that is liable to exert a tensile stress on elastic fibers or induce movements of fluid in the cutaneous tissue and thus change the mechanical parameters, resulting in a truncated measurement.

The technical problem to be solved by the inventors thus consisted in designing a measurement device able to evaluate the mechanical properties of the skin while remaining free from all the parameters interfering with measurement.

Thus the object of the present invention is a device for measuring the mechanical properties of the skin characterized in that it comprises:

a) a pneumatic assembly for delivering compressed air to apply a stress to the surface of the skin undergoing measurement,

b) an electronic assembly for contact-free measurements on said surface of the skin subjected to the stress, and

c) a control software for controlling the electronic assembly and for processing measurement data in order to deduce the mechanical properties of the skin.

More particularly, the pneumatic assembly consists of an air compressor which supplies a nozzle via a pneumatically operated switch, a flow rate regulator and a pressure regulator.

The electronic assembly comprises:

a laser-diode laser-line generator for projecting a laser line on said skin surface, and

a CMos or CCD image sensor for recording the position of the points of said laser line, which together constitute a laser-line profilometer.

According to a second aspect, the present invention is directed to a process for measuring the mechanical, surface and volume properties of the skin characterised in that it comprises the following steps:

a) applying a deforming stress to the skin surface under measurement by using an air jet with adjustable pressure and flow rate,

b) projecting a laser line on the surface of the skin deformed by said stress,

c) recording the position of the laser line points at a determined frequency and without contact with said skin surface,

d) processing the data recorded in step c) using a software to deduce the mechanical, surface and volume properties of the skin.

The above features and advantages will be clearer in the following description and attached drawings, in which:

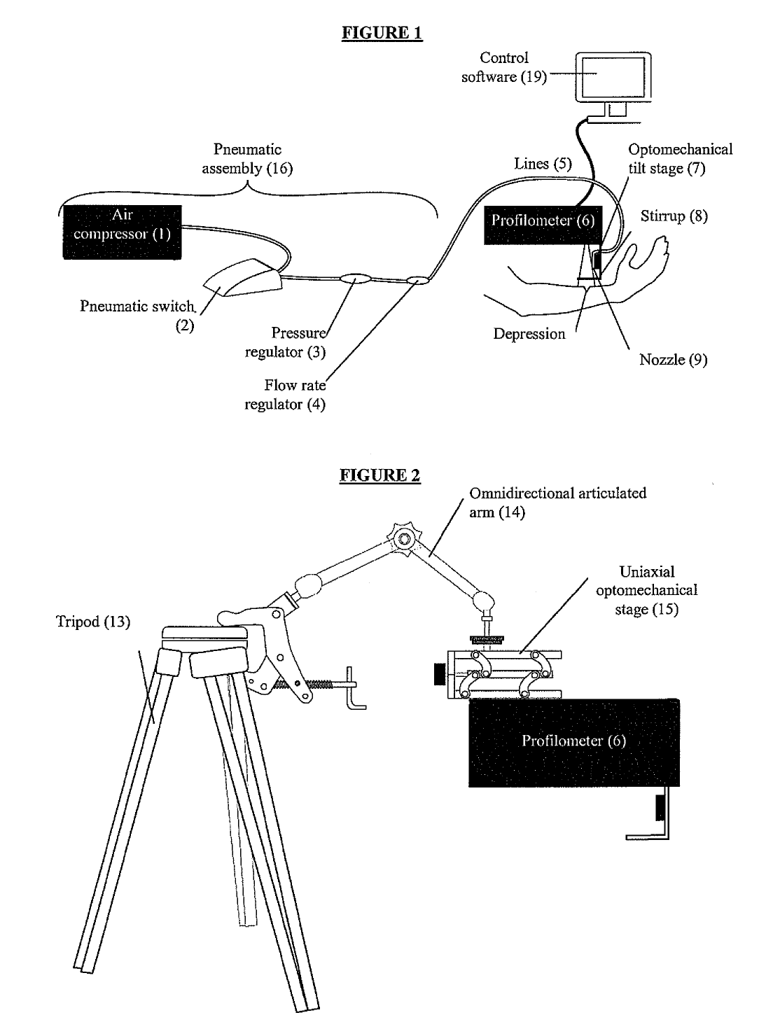

FIG. 1 shows a side view of the device (the tripod, articulated arm and uniaxial optomechanical stage being not shown).

FIG. 2 shows a side view of the profilometer fixation means.

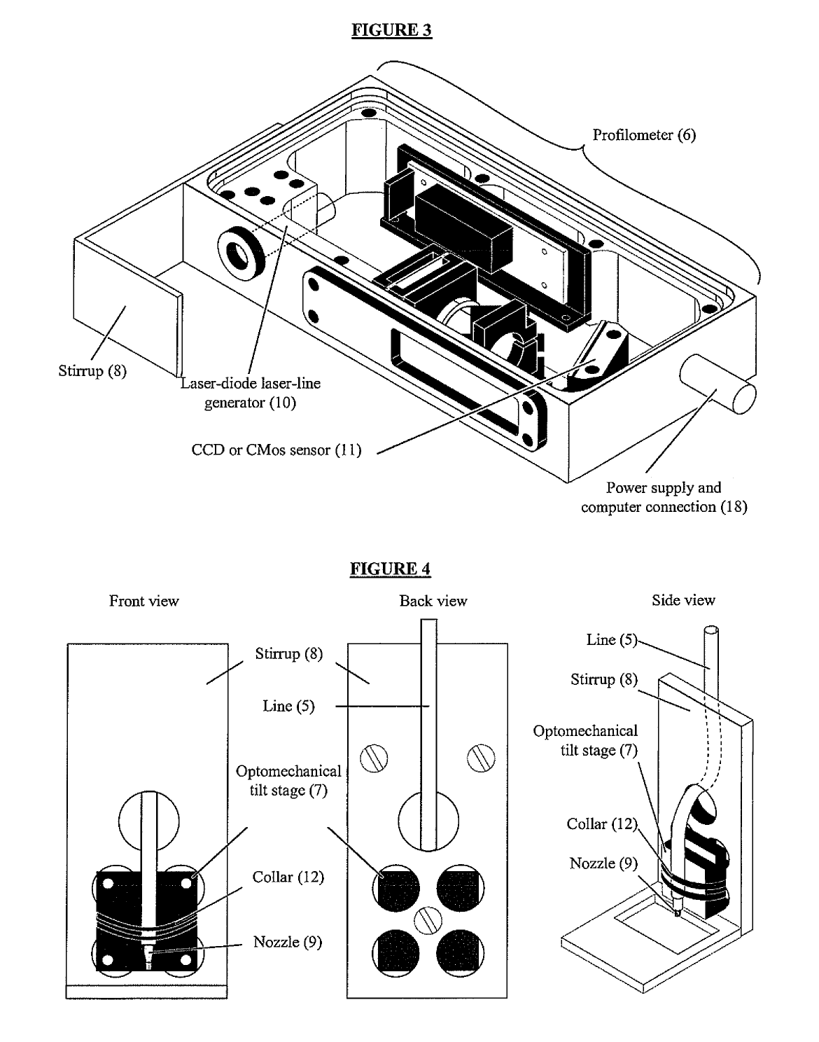

FIG. 3 shows an interior side view of the profilometer.

FIG. 4 shows a front, back and perspective side views of nozzle attachment to the stirrup.

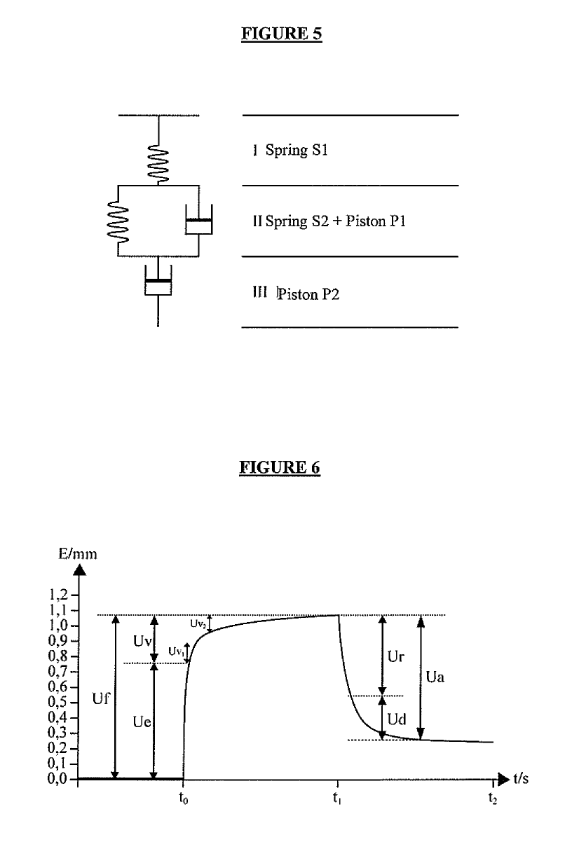

FIG. 5 shows the scheme of Burger's model.

FIG. 6 shows the change in displacement of a system (such as the skin) whose behavior can be modeled using Burger's model.

FIG. 7 shows the application of the compressed air stress to the skin as a function of time (t).

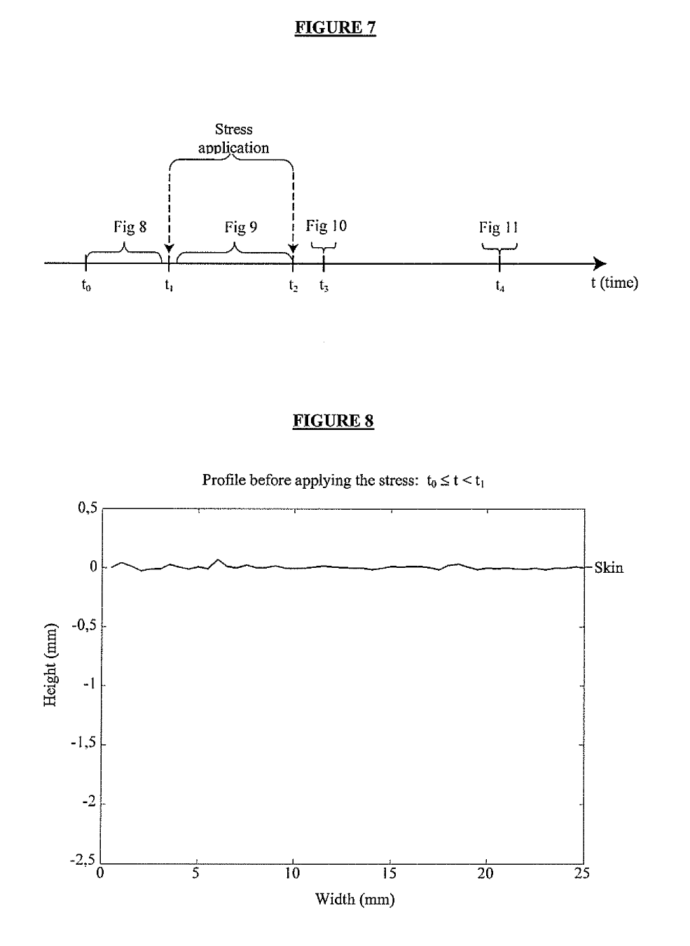

FIG. 8 shows an example of a linear zone measured before applying the stress at a time t such that t₀≦t<t₁.

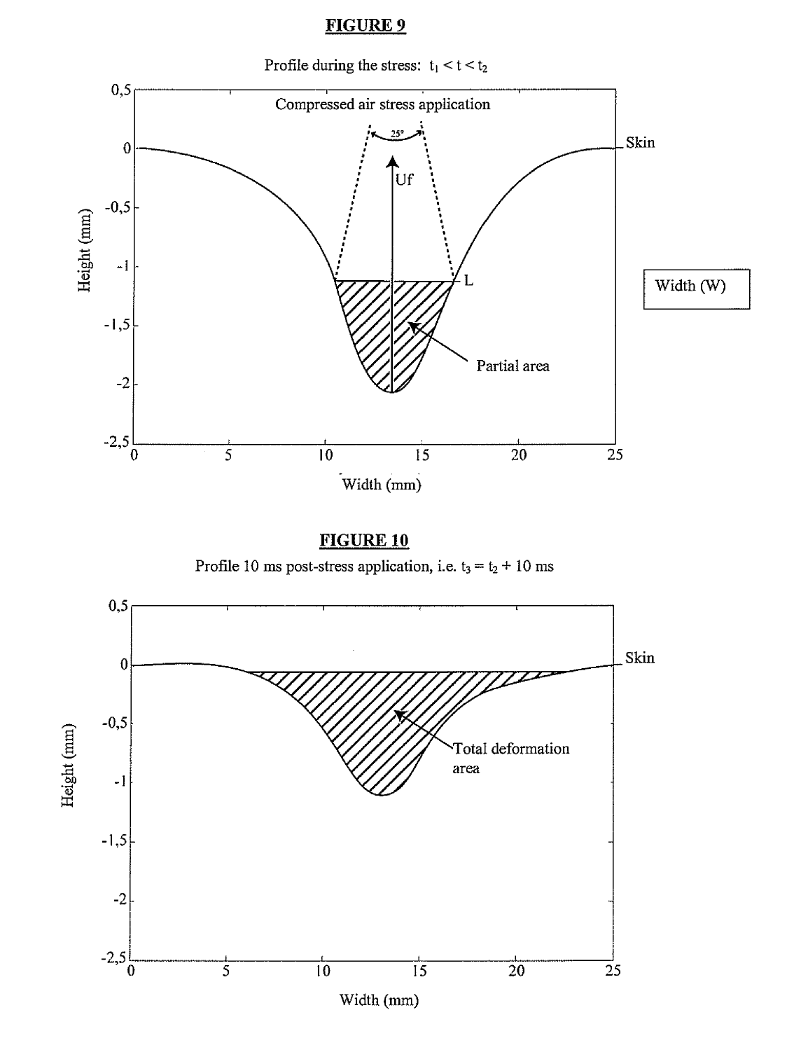

FIG. 9 shows another example of the profile of the same linear zone during application of the stress at a time t such that t₁<t<t₂.

FIG. 10 shows an example of the profile of the linear area 10 ms after applying the stress, i.e. at t₃=t₂+10 ms, the frequency being 100 Hz.

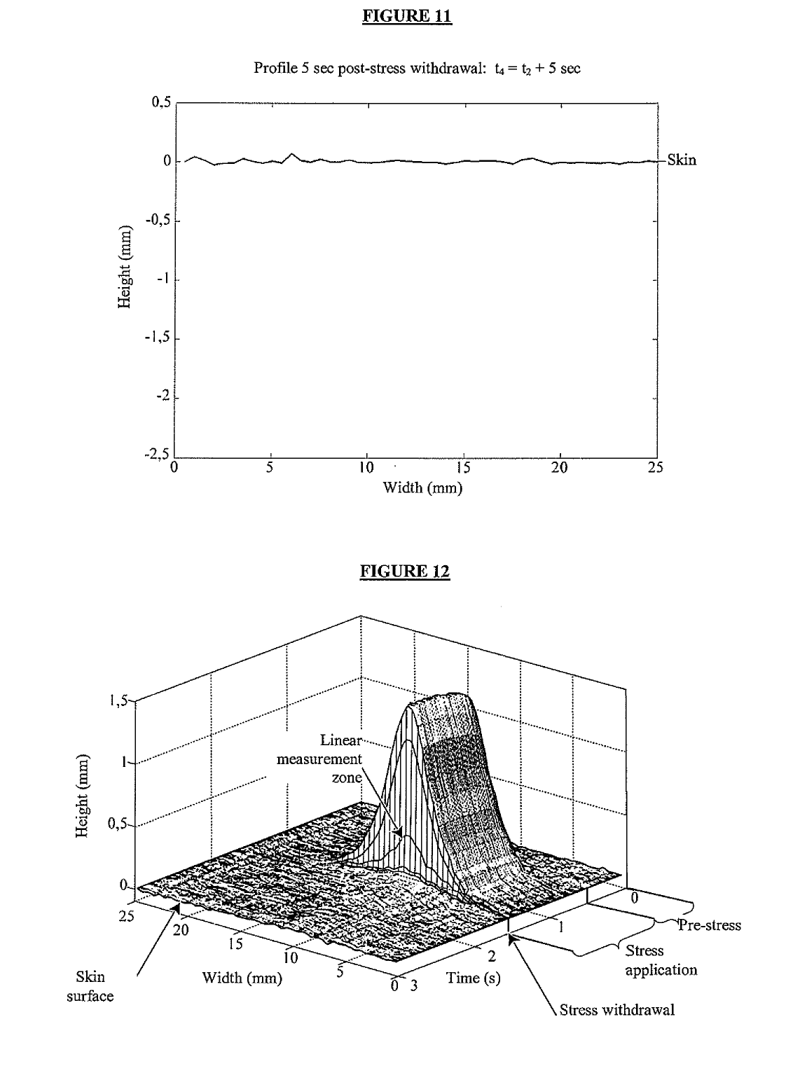

FIG. 11 shows an example of the profile of said linear zone 5 s after applying the stress at t₄=t₂+5 s.

FIG. 12 shows the change in the displacement profile of the linear zone, as a function of time, pre-, per- and post-compressed air stress.

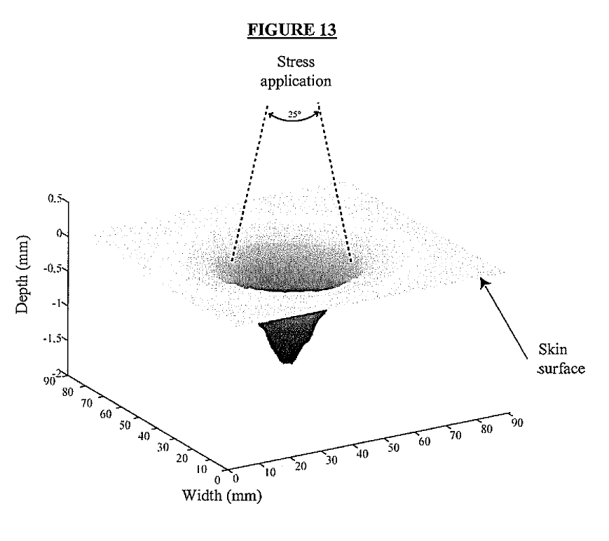

FIG. 13 shows the volume of cutaneous displacement.

More particularly, the device according to the present invention comprises in a laser-diode laser-line generator (10) and a CMos (complementary metal-oxide semiconductor) or CCD (charge-coupled device) (11) sensor that can be combined in a single apparatus in order to constitute a laser-line profilometer (6) able to measure at high frequencies. The expression ‘high frequencies’ refers to frequencies between 50 and 1000 Hz, preferably greater than or equal to 100 Hz, and, in particular, 250 Hz. In the context of the present invention, the expression ‘high frequencies’ indicates a measurement that is repeated in a time interval of between 20 ms and 1 ms, enabling access to continuous measurement.

The optical measurement is non-invasive, fast and very precise, the linearity of the profilometer being less than 15 microns (=μm) and, preferably, about 1 micron. The axial resolution of the profilometer is less than 5 microns and preferably about 1 micron.

The stress is induced on the measurement zone via compressed air delivered by an air compressor (1), pressure and flow rate being variable. In the context of the present invention, an ‘air compressor’ refers to an apparatus designed to increase the air or gas pressure in a container, this term also includes the use of pressurized cylinders.

Said air compressor (1) is connected by a semi-rigid line to a pneumatic switch (2) enabling control of stress application time as well as the profile on the software graphic interface. The switch may consist in a pedal, manual device, or solenoid valve that parameterizes the duration of the stress. According to the invention, the term ‘stress’ refers to the force exerted on the measurement zone per unit area.

In addition, to achieve the present invention, it is important that the flow rate and pressure of the air delivered by the compressor remain stable during measurement. Accordingly, said pneumatic switch is connected to a pressure regulator (3) which is in turn connected to a flow rate regulator (4). To overcome the problem of barometric variation, the pressure regulator (3) includes at least one micrometer screw enabling constant pressure to be maintained. The flow rate regulator (4) also includes at least one micrometer screw. The use of a regulator with micrometer screw is necessary for fine adjustment of the air flow.

The flow rate regulator (4) is then connected to a nozzle (9).

In order for the compressed air delivered by the nozzle to deform the skin at laser line level, an optomechanical tilt stage (7) is used. The optomechanical tilt stage (7) enables fine adjustment of the nozzle angle using a micrometer screw and hence orientation of the compressed air jet on the measurement line. Said nozzle is attached to the stage (7) using a collar (12). The stage is in turn attached to a stirrup (8) by one or several screws. The stirrup is screwed to the profilometer (6). Thus, according to another aspect of the invention, the nozzle is orientable via an optomechanical tilt stage (7).

The various components of the device are connected by semi-rigid lines (5).

Obviously, the order described is in no way limitative and the various components may be interchanged to ensure correct operation of the system.

The distance between the air nozzle and surface measured is between 1 mm and 10 cm or more and preferably about 1 cm. The form of the nozzle may be varied in order to modify the jet impacting the skin. A single nozzle or multiple nozzles may be used.

The present device also includes a uniaxial optomechanical translation stage (15) which is attached both directly to the profilometer (6) and to an omnidirectional articulated arm (14). The expression ‘omnidirectional articulated arm’ is taken to mean a system that enables the profilometer to be oriented in all directions.

Said articulated arm is then fixed to a tripod (13). Obviously, the tripod may be replaced by any other component ensuring satisfactory stability.

The articulated arm positions and fixes the device, then the uniaxial translation stage (15) enables the profilometer to be raised or lowered by vertical movement. The system is positioned above the measurement site so as to maintain a constant distance between the profilometer and the surface under study.

One of the advantages of the invention is thus the measurement without any contact between the device and the zone to which stress is applied. Therefore, the term ‘contact-free’ indicates that the device and components enable measurement and data collection without entering into contact with the zone under evaluation.

The profilometer is also connected to a computer running the software controlling the sensor assembly and recording the data.

The data are obtained by triangulation of a laser line in a system enabling modulation of measurement frequency. The lateral resolution is less than 2 mm and preferably between 10 and 100 μm. The data are transmitted to the computer via the data-acquisition software, and after processed using mathematical algorithms.

The software of the device thus enables high-frequency acquisition of cutaneous profiles and, more particularly, of a linear zone as a function of time.

Thus, the control software measures skin displacement as a function of time pre-, per- and post-stress application.

According to a preferred embodiment, the present device has the advantage of being small, light and thus easily transportable.

The device according to the present invention has a great flexibility; Thanks to the omnidirectional articulated arm (14), all the areas of the body may be evaluated: forehead, face, neck, forearm, hand, bust, abdomen, leg, foot, etc.

In another embodiment, the stress may be induced using any gas from a compressor or pressurized cylinder fitted with a pressure-reducing valve.

Measurement may also be implemented by any system able to measure a profile or full-field measurement, in a static and dynamic manner, on any surface.

More particularly, the present invention may also include an apparatus simultaneously measuring a number of defined points in a measurement zone using chromatic confocal imaging photometry and exploiting the axial chromatism of the lens system.

Accordingly, the present invention may incorporate an apparatus based on that technology such as:

a point sensor with a wide measurement field range from 20 μm to 24 mm. Such sensor enables high axial precision, generally less than a micrometer. For example, the axial precision of an optical wand with a measurement field of one centimeter is 600 nanometers. The measurement frequently may reach 40 kHz;

a line sensor reporting a surface profile in a single measurement. Such device enables contact-free surface topography and 3D-measurement. The measurement line is subdivided into 180 points and the measurement frequency is up to 1.8 kHz;

chromatic confocal field sensor obtained by adding a scanning mirror in the line sensor optical head. Two models are available:

measurement field: 1.8×1.8 mm, 500 μm of measurement field, axial resolution of 125 nm and axial precision of 500 nm

measurement field: 44.75×44.75, 2 mm of measurement field, 500 nm of axial resolution and 2.5 μm of axial precision.

These two sensors enable one measurement every 130 ms.

In another aspect of the invention, the currently known devices in the cosmetic field and, more particularly, the devices used to measure the mechanical properties of the skin do not enable two-or three-dimensional measurements of a surface subjected to stress at high frequencies.

The devices, which are familiar to professionals specialized in cosmetic devices measuring the properties of the skin, are able to make high-frequency measurements only for a point located in the measurement zone. Examples include the Cutometer. There are also devices enabling measurement in two (=linear zone) or three dimensions but for a given time, for example the fringe-projection method. However, these devices do not enable high-frequency two- or three-dimensional measurements of a surface subjected to stress.

The present invention thus consists in a device which makes high-frequency measurements of a linear zone.

Thus, the present invention refers to a device able to make high-frequency measurements of the evolution of a same linear zone subjected to stress. More particularly, the present invention uses a laser-diode laser-line generator (10) which projects a laser line on the surface whose topography is to be determined At the same time, the compressor delivers air to the line in order to deform the measurement zone.

The laser line is reflected on the CMos or CCD sensor (11) which divides it into a number of points proportional to the lateral resolution (laser line width/lateral resolution). By triangulation, the profilometer measures, in an absolute manner, the height of each point on the line in order to deduce, via the data-processing software, the mechanical, surface and volume properties of the measured zone.

The profilometer has a lateral resolution of less than 2 mm and axial resolution of less than 5 microns, and preferably around 1 micron or less. The measurement frequency is preferably greater than 50 Hz, for example 100 Hz, and may reach 1000 Hz. Thus, the position of the points on the laser line projected on the skin surface to be measured is recorded at a frequency of between 50 and 1000 Hz, and preferably about 250 Hz.

Therefore the interest of the laser line resides in the ability to obtain multipoint information (linear zone) over several centimeters within a single measurement and thus to avoid the artifacts that may be associated with a prompt measurement.

In order to extract the mechanical, surface and volume properties of the material, a number of adjustments and calculations using mathematical algorithms are necessary. Scientific calculation software enables processing of such data.

The measurements are thus transferred to EXCEL or MATLAB for processing. The EXCEL or MATLAB data are the measurements for each point of the linear zone for a given time (FIGS. 8, 9, 10 and 11) and for all times (FIG. 12). The number of profiles per second depends on the acquisition frequency. At first, the data are rectified for exploitation. Since the system is very precise (linearity of plus or minus 15 μm), the slightest movement of the subject falsifies the results.

These points subsequently enable three-dimensional graphic exploitation and determination of the magnitude of the skin displacements.

In a single measurement, it is thus possible to obtain a linear zone profile, i.e. a two-dimensional spatial measurement of the linear zone at a given time (FIGS. 8, 9, 10 and 11). The measurement is the absolute measurement of the distance between the laser diode and surface observed.

To facilitate comprehension of the invention, FIGS. 7, 8, 9, 10, 11 and 12 are given as examples and explained below.

FIG. 7 shows application of the compressed air stress on the skin as a function of time during a procedure at a frequency of 100 Hertz (Hz). FIGS. 8, 9, 10 and 11 are dependent on FIG. 7 and show the profiles measured at the various times. More particularly, FIG. 8 shows an example of the profile of a linear zone undergoing measurement before applying a stress, i.e. at a time t such that t₀≦t<t₁. FIG. 9 shows another example of the profile of the same linear zone during stress application, i.e. at a time t such that t₁<t<t₂. FIG. 10 also shows an example of the profile of the linear zone at t₃=t₂+10 ms at a frequency of 100 Hz. FIG. 11 shows an example of the profile of said linear zone 5 s after stress applying at t₄=t₂+5 s.

In addition, the various profiles obtained during the procedure described in FIG. 7 showing the same linear zone subjected to a constant stress with measurement every 10 ms (pre-, per- and post-stress) are shown in FIG. 12. FIG. 12 thus shows a breakdown of the cutaneous displacement of the same linear zone pre-, per- and post-application of the stress and every 10 ms.

Thus, one of the advantages of the present invention is the ability to measure the displacement profile of a linear zone subjected to compressed air stress with high-frequency measurement between 50 and 1000 Hz and preferably at about 250 Hz.

In another aspect of the invention, the device has the advantage of high-frequency measurement of various parameters directly related to the process of skin aging. Those parameters were difficult to obtain at high frequency before implementation of the present invention. The present invention enables measurement, at a frequency of between 50 and 1000 Hz, and preferably about 250 Hz, of the surface properties, tissue cohesion and volume properties of a measurement zone subjected to a stress at a given time.

### Measurement of Surface Properties and Tissue Cohesion (FIGS. 9, 10)

According to the present invention, it is possible to calculate a certain number of characteristics directly related to skin aging.

In fact, the scientific calculation software enables calculation of the area of displacement of each profile measured at high frequency (FIG. 9). It is thus possible to study, at high frequency, the change over time of the areas of the same zone measured pre-, per- and post-application of the stress.

It is also possible to investigate a fraction (L) of the maximum height (Uf) of a profile at a given time (FIG. 8).

With the present invention it is possible to obtain and thus to study a new parameter, which is the time course of tissue cohesion. Tissue cohesion directly reflects the ability of the material under study to absorb impacts. More particularly, when a force is exerted at constant pressure on young skin and on older skin, the young skin distributes the stress more widely than the older skin. The mechanical qualities of young skin enable diffusion of the incident pressure over a greater area than will do an older skin. By analogy, the mechanical properties of young skin may be compared to the mechanical properties of a trampoline while the mechanical properties of older skin may be compared to the mechanical properties of modeling clay. When a force is exerted on mature skin, the cutaneous tissue assumes a shape induced by the stress, in particular due to a decrease in the density of the extracellular matrix. The older the skin is the longer it takes to return to its initial shape. In contrast, young skin diffuses the stress experienced over all its surface and thus returns to its initial shape almost immediately: the means of decreasing the pressure of an impact consists in increasing the area subjected to the impact.

Tissue cohesion is a function of two parameters: Uf, the maximum amplitude of displacement, and W, the width of the deformation at a predefined fraction of the height.

Thus, the denser and more cohesive a tissue is, the better it absorbs compressive impacts (axial compression of the tissue). The greater the width is, the greater the distribution of the deformation, the greater the tissue cohesion and the more elastic the skin will be, and, hence, the younger the skin will be.

The present invention thus enables study of the total or partial area of skin displacement for each profile (FIGS. 9 and 10) and hence the evolution, at high frequency, of these areas. In addition, the present invention enables obtaining a new parameter for measurement of the properties of the skin: the evolution at high frequency of tissue cohesion.

### Volume Properties:

The use of the stress device without contact on the measurement zone enables imaging, quantification and calculation of the amplitude of displacement in the three axes of an orthonormal system (x, y, z) and as a function of time.

The present invention is based on the hypothesis that the deformation is isotropic (since the stress is symmetrical). It is thus possible to extrapolate the volumes by 180-degree rotation of the profiles. A hollow derived from displacement of the measurement zone is thus obtained and represents the real calculated image of the volume of skin displacement.

As was the case for areas, the present invention enables obtaining the total volume or a fraction of that volume together with the volume of a profile at a given time or the evolution of the volume of skin displacement and at high frequency. Thus another advantage of the present invention is to make it possible to monitor the evolution of skin displacement volume, at high frequency. FIG. 13 shows the volume of skin displacement.

### Measurement of Mechanical Properties:

In the context of the present invention, ‘mechanical properties’ means the physical parameters that can be defined from the displacement of a material subjected to a mechanical stress. All deformable materials, including the skin, have elastic, viscoelastic and plastic properties, i.e. a succession of responses to the applied stress.

Thus, the term ‘elasticity’ indicates the non-malleable part of the deformation under stress. The term ‘plasticity’ refers to the malleable part of the deformation under stress.

Burger's model enables the mechanical behavior of a material to be modeled.

FIG. 5 shows a scheme of this model symbolized by an assembly of springs and pistons. FIG. 6 shows the evolution of displacement of a system (such as skin) whose behavior can be modeled using Burger's model in which:

Ue indicates elasticity,

Uv indicates viscoelasticity,

Uf indicates final deformation,

Ur designates immediate recovery,

Ud designates delayed recovery,

Ua designates added recovery.

Under strong and prolonged but reversible stress, the skin undergoes two successive types of elongation:

the Ue phase of immediate extension, considered purely elastic, due to the elastic network of the dermis, to collagen bundles and to the deformability of the epidermis and of the stratum corneum,

the viscosity (Uv) which includes Uv1 (a variable extension (creep)) and/or Uv2 (constant creep) which are due to the basic substance, the difficulty of liquids circulating between dermal fibers and friction between protein structures.

The limits between Ue and Uv1, and Uv1 and Uv2 are imprecise since they depend on the proportions of elastic and viscous resistance in the tissue, which vary depending on the site, age and subject.

When the stress is stopped, the skin immediately retracts (Ur, purely elastic recovery), but in an incomplete manner in accordance with a logic similar to that of extension. More time is required for complete recovery (viscoelastic recovery phase). Recovery is fast in children and slower in the elderly.

In contrast, in the event of important extensions or compressions (injury), the deformation is plastic and irreversible (Agache et al., 2000).

Thus, according to the present invention high-frequency measurement of the elastic, plastic and viscoelastic properties of a linear zone of the skin subjected to stress is possible. In consequence, one of the advantages of the present invention is to enable fine measurement free from the artifacts that may derive from one-dimensional measurements and free from the confounding parameters that would result from direct contact between the system and measurement zone.

Thus, the present device is able to implement high-frequency measurement of a linear zone subjected to a stress while remaining free from all the parameters interfering with measurement. More particularly, the invention enables monitoring and recording using precise, continuous and reproducible measurements of the mechanical, surface and volume properties of skin displacement.

It is thus possible to continuously measure skin displacement and recovery to the equilibrium state in the fields of cosmetics, dermatology, human and animal diseases and/or nutraceuticals. The device enables high-frequency measurement of the evolution of the area, volume and tissue cohesion of a zone subjected to a stress. Thus, the system enables, from a linear zone subjected to a stress, deduction of the surface properties, tissue cohesion and volume properties of the zone under stress.

The device also enables study of the mechanical properties and particularly the elastic, plastic, viscoelastic, tone, fatigue, recovery and suppleness properties of a linear zone of the skin at high frequency. In the context of the present invention, the term ‘tone’ indicates the ability of the skin to rapidly return to an equilibrium state when the stress is withdrawn.

Thus, the present invention enables monitoring of the natural or induced aging of said material. The physiological or pathological changes in the skin related, for example, to natural skin aging (chronological) or induced skin aging (medicinal treatment, ultraviolet radiation, environment, pollution, hygiene products, medical devices, cicatrization, infrared radiation, cold, dry air, foods, toxic substances) or cosmetic treatments can thus be followed up and evaluated. The process is non-invasive and non-destructive.

The device enables monitoring pathological skin in man or animals and monitoring of the course or regression of disease by monitoring the changes in mechanical parameters over time or following topical or oral treatment. The device may be used to diagnose diseases directly or indirectly affecting the mechanical properties of the skin, and, if necessary, to enable orientation toward the treatments most appropriate for the condition of the skin or its characteristics.

The device also enables evaluation, on healthy or pathological skin, of the direct or indirect effect of medical devices or massages on the mechanical properties of the skin associated or not associated with application or intake of a product or use of luminotherapy.

The device also enables evaluation of the direct or indirect modifications of healthy or pathological skin induced by medicinal products, dermatological products, cosmetic products, nutraceuticals, cosmetics (moisturizing products, sunscreens, anti-wrinkle products, firming products) and foods.

According to another aspect, the present invention enables measurement of the evolution of the mechanical, surface and volume properties of all deformable surfaces or materials such as the skin, deformable polymers, foams and elastomers.

The device will also be of value in specialized industries such as the following:
polymer industry: solids, gels and foams;
construction industry: building materials (insulators, anti-vibration products);
medical device industry (e.g. rubber prostheses);
food industry (viscosity of food products);
chemical industry (fertilizers, pesticides);
elastomer industry.

1. Device for measuring the mechanical properties of the skin comprising:
a) a pneumatic assembly (16) for delivering compressed air to apply a stress to the surface of the skin undergoing measurement,
b) an electronic assembly (6) for contact-free measurements on said surface of the skin subjected to the stress, and
c) a control software (19) for controlling the electronic assembly (6) and for processing measurement data in order to deduce the mechanical properties of the skin.

2. Device according to claim 1, wherein the pneumatic assembly (16) for delivering compressed air to apply a stress to the surface of the skin comprises an air compressor (1) supplying a nozzle (9) via a pneumatic switch (2), a flow regulator (4) and a pressure regulator (3).

3. Device according to claim 2, wherein the distance between said nozzle (9) and the surface of the skin subjected to measurements is between 1 mm and 10 cm and preferably around 1 cm.

4. Device according to claim 2, wherein said nozzle (9) is orientable by means of an optomechanical tilt stage (7).

5. Device according to claim 1, wherein the electronic assembly (6) for making contact-free measurements on said skin surface under stress comprises:
a laser-diode laser-line generator (10) for projecting a laser line on said skin surface, and
a CMos or CCD sensor (11) for recording the position of said laser line points, which together constitute a laser-line profilometer (6).

6. Device according to claim 5, wherein the position of the points of the laser line projected on the skin surface under measurement is recorded at a frequency of between 50 and 1000 Hz.

7. Device according to claim 5, wherein the linearity of the profilometer is less than 15 μm, and the axial resolution of said profilometer is less than 5 μm.

8. Device according to claim 5, wherein the lateral resolution of the profilometer is less than 2 mm.

9. A method of measuring at least one mechanical property of skin, comprising utilizing the device of claim 1 for measuring the profile of a linear zone subjected to a stress by compressed air at a frequency of between 50 and 1000 Hz.

10. A method of measuring at least one mechanical property of skin, comprising utilizing the device according to claim 9 for deducing the surface properties, tissue cohesion and volume properties of the zone subjected to a stress.

11. A method of measuring at least one mechanical property of skin, comprising utilizing the device of claim 1 for high-frequency measuring of the mechanical properties and, particularly, the elastic, plastic and viscoelastic properties of a linear zone subjected to a stress.

12. A method of measuring at least one mechanical property of skin, comprising utilizing the device of claim 1 in the fields of cosmetics, dermatology, human health, animal health and/or nutraceuticals.

13. A method for measuring the mechanical, surface and volume properties of the skin comprising:
a) applying a deforming stress to the skin surface under measurement by using an air jet with adjustable pressure and flow rate,
b) projecting a laser line on the surface of the skin deformed by said stress,
c) recording the position of the laser line points at a determined frequency and without contact with said skin surface,
d) processing the data recorded in step c) using a software to deduce the mechanical, surface and volume properties of the skin.

14. The method of claim 13 for use in the fields of cosmetics, dermatology, pathology, nutraceuticals and veterinary health.

15. The method of claim 13 for monitoring the natural or induced aging of skin.

16. The method of claim 13, wherein the method is non-invasive and nondestructive.

17. A portable, contact-free device for measuring tissue cohesion of skin, comprising:
a) a pneumatic assembly for delivering compressed air to apply a stress to the surface of the skin undergoing measurement;
b) a laser-line profilometer for making contact-free measurements on said skin comprising:
i) a laser-diode laser-line generator for projecting a laser line on said skin surface, and
ii) a CMos or CCD sensor for recording the position of said laser line points; and
c) a control software for controlling the electronic assembly and for processing measurement data in order to deduce the tissue cohesion of the skin.

18. The contact-free device of claim 17, wherein the measurement comprises triangulation of a laser line.

19. The contact-free device of claim 17, wherein the device is capable of making three-dimensional determinations of skin displacement.

20. A non-invasive, non-destructive method of measuring the natural or induced aging of skin, comprising utilizing of the portable, contact-free device of claim 17.
